(19) United States
(12) Patent Application Publication
(10) Pub. No.: US 2009/0017113 A1
(43) Pub. Date: Jan. 15, 2009

(54) DULOXETINE FORMULATIONS

(76) Inventors: Niels J. Osinga, Nijmegen (NL); Dirk Pamperin, Nijmegen (NL)

Correspondence Address: SYNTHON IP INC, 7130 HERITAGE VILLAGE PLAZA, STE 202, GAINESVILLE, VA, 20155, US

(21) Appl. No.: 12/171,703

(22) Filed: Jul. 11, 2008

Related U.S. Application Data
(60) Provisional application No. 60/949,834, filed on Jul. 13, 2007.

(51) Int. Cl. A61K 9/58; A61K 9/22; B05D 7/00; A61P 25/24; A61P 25/20

(52) U.S. Cl. 424/462; 427/214; 424/468

(57) ABSTRACT

Duloxetine pellets having an enteric coating containing a polymethacrylate polymer can be formed with desirable release rates/profile and stability.

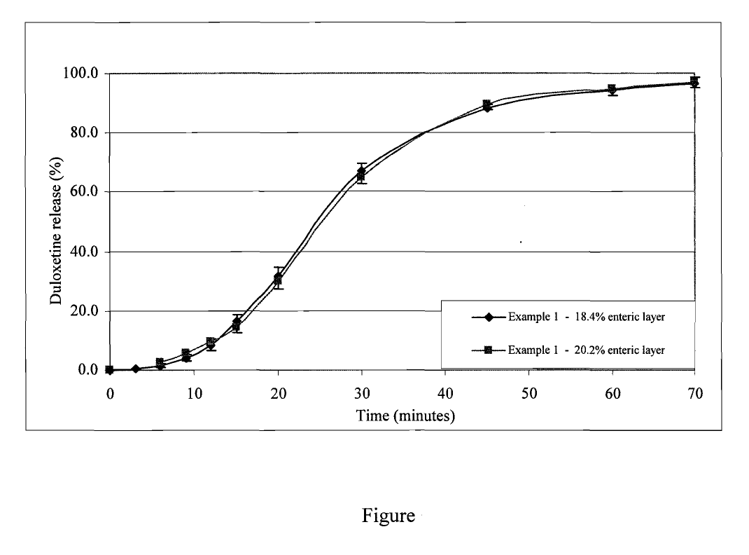

This application claims the benefit of priority under 35 U.S.C. § 119(e) from prior U.S. Provisional Application Ser. No. 60/949,834, filed Jul. 13, 2007, the entire contents of which are incorporated herein by reference.

### BACKGROUND OF THE INVENTION

The present invention relates to an improved pharmaceutical dosage form of duloxetine and its use as a medicament.

Duloxetine is a pharmaceutically active compound useful as an antidepressant. See, for example, Wong et al., Neuropsychopharmacology, 8, 23-33 (1993), where the compound is named by its research number LY248686.

Duloxetine is (+)-N-methyl-3-(1-naphthalenyloxy)-2-thiophenepropanamine, and is commonly used in pharmaceutical compositions as its hydrochloride salt. In this document, the word “duloxetine” will refer to the specific enantiomer just named.

The marketed pharmaceutical dosage form of duloxetine sold in the US by Eli Lilly & Co. under the brand name Cymbalta® is a capsule comprising a plurality of enteric coated pellets containing duloxetine hydrochloride.

Enteric pharmaceutical formulations are manufactured in such a way that the product passes unchanged through the stomach of the patient, but dissolves and releases the active ingredient after it leaves the stomach and enters the small intestine. Such formulations conventionally are in tablet or pellet form, where the active ingredient is in the inner part of the tablet or pellet and is enclosed in a film or envelope, i.e., the “enteric coating”, which is insoluble in acid environments, such as the stomach, but is soluble in near-neutral environments such as the small intestine.

The need to formulate duloxetine in an enteric formulation is due to the poor stability characteristics of duloxetine in acidic solutions. The duloxetine molecule decomposes easily in an acidic environment upon formation of a highly toxic naphthol moiety.

When a pharmaceutical dosage form has been orally taken and before it reaches the intestines it normally resides in the stomach for a period of 0.2-2 hours. According to “Innovations in drug delivery” (ISBN 90-73520-06-1) by C. G. Wilson and N. Washington, pg 42-56, typically the resting gastric pH of a normal healthy subject is around 1.8. Meals markedly alter the pH which can increase to 3-5 after eating, whereafter the resting gastric pH is obtained again. As can be seen in the same chapter gastric emptying of pellets from the stomach to the intestines can take up to 3 hours for pellets and up to 9 hours for large non-disintegrating tablets, depending on the content of the stomach.

The EP 693282 (and corresponding U.S. Pat. No. 5,508,276) teaches that an enteric coated pellet formulation of duloxetine was more desirable than an enteric coated tablet, based on bioavailability studies which showed that more consistent plasma profiles were obtained after pellet administration.

Furthermore, the EP 693282 teaches that certain difficulties arose in preparing conventional enteric coated formulations. Most importantly, duloxetine was found to react with many enteric coatings; forming an insoluble coating in some cases. Because of this unexpected cross-reactivity, formulations using an enteric coated pellet were found to have a disadvantageous drug-release profile and low bioavailability.

Further, it was found to be particularly difficult to prepare an enteric formulation with higher levels of drug loading which did not allow some release of duloxetine in the acid environment, thus creating a danger that some drug would be released in the stomach, contrary to the desired safe method of administration.

As a solution of the above problems, the EP 693282 offers an enteric coated pellet comprising (a) a core consisting of duloxetine [hydrochloride] and a pharmaceutically acceptable excipient; (b) an optional separating layer; (c) an enteric layer comprising hydroxypropylmethylcellulose acetate succinate (HPMCAS) and a pharmaceutically acceptable excipient; and (d) an optional finishing layer.

The HMPCAS polymer was selected, in part, for use in the enteric coating because of its small number of carboxylic acid groups per unit weight or repeating unit of the polymer. The HPMCAS has been defined as containing not less than 4% and not more than 28% of succinoyl groups, which are the only free carboxylic groups in the compound. It is commercially available (under brand name AQOAT™ made by Shin-Etzu) in two particle size grades and three molecular weight ranges.

In the case of coating HPMCAS from a water-based system, the dissolution of the polymer in the aqueous solvent can be obtained by neutralizing the polymer, preferably with ammonia. For example, operation with from about 25% to about 100% neutralization has been preferred in the above document. The coating process is, however, somewhat difficult.

In an article of P. J. Jansen et al. (J. Pharm. Sci., vol. 87 (1) p. 81-85, (1998)) it is explained that duloxetine reacts with polymer degradation products or residual free acids in hydroxypropyl methylcellulose acetate succinate (HPMCAS) and hydroxypropyl methylcellulose phthalate (HPMCP) in dosage formulations to form succinamide and phthalamide duloxetine derivatives, respectively, as impurities.

It has been proposed that polymer-bound succinic and phthalic substituents can be cleaved from the polymer resulting in the formation of either the corresponding free acids or the anhydrides. These can react with duloxetine to form the succinamide and phthalamide impurities.

It has been postulated that the reaction between duloxetine and the enteric coating material is enabled by the migration of either (1) the free acid or (2) the parent drug through the formulation. The formation of impurities was minimized by increasing the thickness of the physical barrier (subcoat) separating the enteric coating from the drug.

It has been furthermore stated that the succinamide and phthalamide impurities were actually detected in enteric coated products upon storage in pharmaceutically relevant storage conditions.

The danger of forming the succinamide and phthalamide impurities is pharmaceutically relevant and forms a serious disadvantage of the HMPCAS coating.

In practice, the use of the HPMCAS as a coating material also faces several technological problems. As seen from the Examples in EP 693282, the coating by HPMCAS must be performed upon cooling and the HMPCAS must be neutralized by ammonia to provide an aqueous solution. In addition, the use of a special dual channel nozzle is suggested to avoid clogging (gelation) of the HPMCAS in the tubing of the coater.

WO 2005/108386 describes duloxetine free base and novel polymorphic forms thereof. Also some pellet formulations containing duloxetine base or duloxetine HCl are described in examples 6-9. The disclosed formulations all have a seal coating between the duloxetine containing layer and an enteric coating layer. The enteric coating shown in these examples comprises approximately 7-14 wt % based on the total weight of the bead/pellet and contains the polymer Eudragit L 100-55. However, these formulations do not exhibit the desired release rate of duloxetine. Similar formulations have been disclosed in WO 2007/139886.

It would be advantageous to find a useful enteric coating for a duloxetine composition, particularly duloxetine pellets. It would also be desirable to find an effective enteric coating that avoided or minimized any of the above-noted disadvantages.

### SUMMARY OF THE INVENTION

The present invention relates to pharmaceutical dosage forms of duloxetine hydrochloride. A first aspect of the invention relates to a pharmaceutical dosage form comprising a plurality of pellets, wherein each pellet comprises:

i) a water soluble pellet core with a diameter of 600-1000 micrometer;

ii) a drug layer covering the core, comprising duloxetine or a pharmaceutically acceptable salt thereof, especially duloxetine hydrochloride, and a binder, which preferably is methyl cellulose, which layer typically constitutes 24-32 wt % of the total weight of the pellet composition;

iii) a separating layer covering the drug layer, comprising a binder and a pore forming component, which layer constitutes 3-12 wt % of the total weight of the pellet composition; and

iv) an enteric coating layer covering the separating layer, comprising a pharmaceutically acceptable acid resistant polymethacrylate polymer, which typically constitutes 18-27 wt % of the total weight of the pellet composition.

A second aspect of the invention relates to the use of the pharmaceutical dosage form defined above as a medicament, preferably for the treatment of stress urinary incontinence, major depressive disorder, general anxiety disorder or neuropathic pain.

Another aspect of the invention relates to a process, which comprises:

coating a pellet core having a diameter of 600-1000 micrometer, with a drug layer, comprising duloxetine or a pharmaceutical acceptable salt thereof and a binder;

coating said drug layer with a separating layer, comprising a binder and a pore forming component, wherein said separating layer constitutes 3-12 wt % of the total weight of the pellet composition; and

coating said separating layer with an enteric coating layer, comprising a pharmaceutically acceptable acid resistant polymethacrylate polymer; wherein said enteric coating layer is coated as a solution in a non-aqueous solvent onto said separating layer.

### BRIEF DESCRIPTION OF THE DRAWING

The FIGURE represents the dissolution results for Example 1.

### DETAILED DESCRIPTION OF THE INVENTION

The present invention relates to pharmaceutical formulations of duloxetine having a defined enteric coating. Unless otherwise stated the expressions of percentage will be in weight percentage.

The pharmaceutical dosage forms of the invention comprise a plurality of pellets wherein each pellet comprises a pellet core, said core being successively layered by a drug layer, a separating layer and an enteric coating layer. Additional layers are possible, such as a finish layer over the enteric coating.

The pellet core of the formulation of the invention is a spherical core (spherical bead) that may be made by pelletizing of pharmaceutically acceptable water soluble materials such as sugars or starches or mixtures thereof in a manner know by a skilled person in the art. Preferably the pellet core may be sugar spheres on which the drug layer of duloxetine hydrochloride may be applied on a manner known by a skilled person in the art. The diameter of the spheres is typically 600-1000 micrometers, preferably the diameter of the spheres is 600-710 or 710-850 micrometers for to obtain the optimal dissolution profile. Typically the pellet core constitutes 35-50 wt % of the total weight of the pellet, preferably the pellet core constitutes 40-45 wt % of the total weight the pellet.

The pellet core is surrounded by a drug layer. The drug layer comprises duloxetine hydrochloride or another water soluble salt of duloxetine, together with a pharmaceutically acceptable binder.

Typically the binder is a hydrophilic polymer and most preferably it is methyl cellulose. Alternatively or in addition thereto, other polymers such as PVP, starch, hydrophilic cellulose derivatives (such as HPMC), and hydrophilic acrylate or methacrylate polymers may be used. The drug layer typically constitutes 24-32 wt % of the total weight of the pellet, wherein the relative amount of the hydrophilic polymer, particularly methyl cellulose, may be 20-40% of the drug layer.

The pellet core comprising the drug layer is surrounded by a separating layer that separates the drug layer from the enteric coating layer. The functions of the separating layer are to provide a smooth base for the application of the enteric coating layer, to prolong the pellet's resistance to the acidic conditions, to improve the drug stability by inhibiting any interaction between the drug and the enteric polymer in the enteric layer, and to improve stability by protecting the drug from light exposure.

It is advantageous that the separating layer constitutes 3-12 wt % of the total pellet weight.

Typically such separating layer comprises a binder and a pore forming agent. Preferably the binder has gel-forming properties after contact with water. A useful binder is, for instance, HPMC such as the commercially available Methocel E5™, or a methyl cellulose. Generally the binder constitutes 40-60 wt % of the total weight of the separating layer.

The pore forming agent is a component which is well soluble in water. Preferably such a pore forming agent is a sugar such as sucrose or a polyvinylpyrrolidone and preferably constitutes 10-40 wt % of the total weight of the separating layer. To counteract the tackiness of such substances, an inert anti-tacking agent may be added to the separating layer, e.g. talc.

The pellet comprising the core, drug layer and separating layer is surrounded with an enteric coating layer. The function of the enteric coating layer is to obtain a release of the active ingredient in the intestines instead of the stomach.

The enteric coating layer in the pellets of the present invention comprises the acid resistant polymethacrylate polymer defined by the current US Pharmacopoeia as “Methacrylic acid copolymer.” Examples include Eudragit L and Eudragit S polymers, which are elaborated, inter alia, in the Handbook of Pharmaceutical Excipients 3rd Ed (Arthur H. Kibbe Ed.). In general the polymethacrylates are copolymers formed of methacrylic acid and one or more of ethyl acrylate, methyl acrylate, methyl methylacrylate, and ethyl methylacrylate. The pharmaceutically acceptable acid resistant polymethacrylate is typically an Eudragit L-type polymer. It may be applied in a manner known by a skilled person in the art or as described in one of the examples of this document. The Eudragit L polymer used to form the liquid coating composition can be supplied as a powder such as Eudragit L 100 or as a water based suspension/emulsion such as for example Eudragit L30D55. Generally the pharmaceutically acceptable acid resistant polymethacrylate polymer constitutes 40-70 wt % of the total weight of the enteric coating layer.

Preferably, a plasticizing agent may be added to the enteric coating layer composition prior to the coating such as for instance a triethylcitrate or dibutylsebacate in an amount as known by a skilled person in the art.

Furthermore, the enteric coating layer composition may comprise inert anti-tacking agent(s), e.g. talc., and/or an antifoaming agent.

In general, no basic agent that could neutralize free carboxylic groups of the polymethacrylate polymer is required in the composition of the enteric coating layer.

The above defined acid resistant polymethacrylate polymer material has several advantages:

a) Lower Degree of Cross-Reactivity with Duloxetine Upon Formation of Succinamide or Phthalamide Impurities.

The cross-reactivity of duloxetine with an enteric polymer such as HPMCAS, HPMCP or PVAP is apparently based on the reaction with free acids or acid anhydrides originating from the polymer. The high molecular mobility and reactivity of these small molecules might have attributed to the product instability.

b) Intrinsically Strong Enteric Polymer

In the case of duloxetine, a strong (i.e. low permeable) enteric coating layer is of crucial importance as the duloxetine easily hydrolyses in an acidic environment upon releasing the toxic naphthol. Therefore, it is generally required for duloxetine-containing formulations that any release of the naphthol in the gastric phase of dissolution testing (2 hrs in 0.1 N HCl) be below 1%.

The relative mass of the polymethacrylate polymer-based enteric coating layer is of importance in determining the dissolution rate of duloxetine in intestinal fluid. From the administrative and therapy point of view, it is desired that at least 75% of duloxetine is released within 45 minutes in the intestinal fluid (which fluid may be simulated by in vitro tests in simulated intestinal fluid dissolution medium pH 6.8 as known from the Pharmacopeias), after prior contact with a stomach fluid (which fluid may be simulated by in vitro tests in 0.1 N HCl [for fasted state of stomach] or by a buffer pH 4.5 [for fed state of stomach]).

If the mass of the enteric coating of the pharmaceutical dosage form of the invention is too high, e.g., more than 40% and often higher merely than 27 wt % of the total weight of the pellet, the release rate of the duloxetine in the (simulated) intestinal fluid is decreased.

Quite surprisingly, when the mass of the enteric coating layer according to the invention is too low, the release is also decreased. Specifically, in this condition the release in the intestinal fluid is not immediate but instead has an undesirable lag time that decreases the overall amount of the released duloxetine.

As a result, the enteric coating layer of the pellet of the invention comprising a polymethacrylate polymer is generally about 10 to 40 wt %, but preferably from 18 to about 27 wt % based on the total weight of the pellet. In some embodiments the mass of the enteric coating layer is about 20-26% and in any embodiment the polymethacrylate polymer is typically a methacrylic acid and ethyl acrylate copolymer. The weight percentages are intended to be calculated on a dry pellet basis.

It has also been found that the coating technique/conditions can affect the performance of the enteric coating layer, as explained more fully hereinafter. Accordingly, it is preferred in some embodiments that the enteric coating layer be one that was formed using a non-aqueous liquid in the coating process. A “non-aqueous” liquid is one that contains at least 50% by volume of a solvent other than water and generally comprises an alcohol, e.g., C1-C4 alcohol, or a mixture of alcohol and water. Moreover, dissolving the polymer into the non-aqueous liquid to form a solution, as opposed to a suspension, is a further preferred technique. An enteric coating layer made by such a non-aqueous liquid can provide reduced lag time, even though the liquid is removed from the pellet, and is thus a preferred enteric coating layer.

Separately from the basic components disclosed above, the pellets of the present invention may also comprise a finishing layer. A finishing layer over the enteric layer is basically not necessary from the functional point of view, but it may be used particularly for to improve the elegance of the product and its handling, storage and machinability properties.

For example, a thin layer of a wax or a polymeric material such as hydroxypropylmethylcellulose, polyvinylpyrrolidone and the like, in an amount such as from a few tenths of % up to about 3%, may be applied. The polymeric material may also carry a suspension of an opacifier, a bulking agent such as talc, or a coloring material, particularly an opaque finely divided color agent such as red or yellow iron oxide. Such a layer quickly dissolves away in the stomach, leaving the enteric layer to protect the duloxetine, but provides an added measure of pharmaceutical elegance and protection from mechanical damage to the product.

Finishing layers to be applied to the present product are of essentially the same types commonly used in pharmaceutical science to smooth, seal and color enteric products, and may be formulated and applied in the usual manners.

The pellets of the present invention can be made by known and conventional techniques. In general, the layers are sequentially coated with the aid of a coating liquid and then dried to remove the liquid. A typical process comprises coating a pellet core having a diameter of 600-1000 micrometer, with a drug layer, comprising duloxetine or a pharmaceutical acceptable salt thereof and a binder; coating the drug layer with a separating layer, comprising a binder and a pore forming component; and coating the separating layer with an enteric coating layer, comprising a pharmaceutically acceptable acid resistant polymethacrylate polymer. In a preferred process the enteric coating layer is coated using a non-aqueous liquid and more preferably as a solution in a non-aqueous solvent onto said separating layer. The non-aqueous liquid or solvent, e.g., an alcohol or alcohol/water mixture, can provide a more advantageous enteric coating layer in terms of initial release or lag time. The amount of water in the non-aqueous liquid or solvent is typically less than 20%, more typically less than 10%.

The pellets can be filled into a capsule or compressed into a tablet to obtain a pharmaceutical dosage form comprising a plurality of pellets which contains an amount of 0.1-100 mg of duloxetine calculated as the free base, per dosage form. Preferably the pharmaceutical dosage form is a capsule and contains 20, 30 or 60 mg duloxetine calculated as the free base.

The pharmaceutical dosage form comprising the plurality of pellets according to the invention desirably exhibits a dissolution release profile of duloxetine of at least 75% within 45 minutes in simulated intestinal fluid (buffer pH 6.8) using USP [symbol] 711 [symbol] , Apparatus 1 method at 100 rpm after having been contacted for 2 hours in simulated gastric fluid (0.1 N HCl) using USP [symbol] 711 [symbol] , Apparatus 1 method at 100 rpm.

The pharmaceutical dosage form with the plurality of pellets of the present invention also desirably exhibits a dissolution release profile of duloxetine of at least 75% within 45 minutes in simulated intestinal fluid using USP [symbol] 711 [symbol] , Apparatus 1 method at 100 rpm after having been contacted for 3 hours in a buffer of pH 4.5 using USP [symbol] 711 [symbol] , Apparatus 1 method at 100 rpm.

The dosage form of the present invention should have sufficient gastric resistance. During the dissolution test of the pharmaceutical dosage form in simulated gastric fluid under the above conditions, preferably less than 1% of the naphthol impurity is formed and released. More preferably less than 0.2% of naphthol is released and most preferably less than 0.1% of naphthol is released.

Furthermore, during the dissolution test of the pharmaceutical dosage form of the present invention in the buffer with a pH of 4.5 under the above conditions, less than 10% of duloxetine is generally released. Preferably less than 5% of duloxetine is released and most preferably less than 1% of duloxetine is released.

The buffer with a pH of 4.5 is understood by a person skilled in the art to be simulating a fed state of the stomach.

In conclusion, the dissolution release profile of the dosage form of the invention in the simulated intestinal fluid described above is maintained independently of the nature of the simulated gastric fluid media in which the dosage form was kept prior to the dissolution testing in the simulated intestinal fluid. The pharmaceutical dosage form of the invention thus has no food effect, i.e. the dissolution release profile of the pharmaceutical dosage form is independent if the stomach is fasted or fed. The dosage form of the invention has also sufficient gastric residence both in the fasted and in the fed state of stomach.

The composition and the dosage form of the invention may be used in any duloxetine-treatable disease. Typically the dosage form of the invention may be used for the treatment of stress urinary incontinence, major depressive disorder, general anxiety disorder or neuropathic pain.

The following examples illustrate the invention.

### EXAMPLE 1

Two batches of duloxetine pellets were made.

Composition:

| | | Batch 1 | Batch 2 |
|---|---|---|---|
| | | (%) | (%) |
| Core | sugar spheres 710-850 microns | 46.4 | 45.3 |
| Drug layer | Duloxetine hydrochloride | 21.9 | 21.4 |
| | HPMC Methocel E5 ™ | 8.8 | 8.6 |
| Separating layer | HPMC Methocel E5 ™ | 2.3 | 2.3 |
| | PVP Kollidon K30 | 1.2 | 1.2 |
| | Talc | 1.2 | 1.2 |
| Enteric layer | Eudragit L30 D-55* | 11.4 | 12.5 |
| | Talc | 5.7 | 6.2 |
| | Triethylcitrate | 1.4 | 1.5 |
| | Simeticon | 0.01 | 0.01 |

| | | |
|---|---|---|
| Content of the enteric layer: | 18.4% | 20.2% |

*=as the dry basis

Process:

The pellet batch was prepared in a fluid-bed coating device (Aeromatic-Fielder MP-2/3) by bottom spray and with a Wuster column installed.

Drug layer was applied onto inert sugar beads of a particle size 710-850 microns. The coating fluid was prepared by combining a dispersion of duloxetine hydrochloride in water and a dispersion of Hypromellose in water. The Hypromellose was allowed to hydrate in water for at least one night.

Separating layer was applied onto the so coated beads. The coating fluid was prepared by combining a dispersion of Hypromellose in water with an aqueous dispersion of PVP and talc (prepared by dissolution of povidone in water followed by dispergating talc under mechanical stirring). The Hypromellose was allowed to hydrate in water for at least one night.

Enteric coating layer was applied onto the beads with the above two coatings. The coating fluid was prepared by combining the aqueous dispersion of Simeticone, triethylcitrate and talc with the Eudragit L30 D-55 ready-to-use dispersion.

After the coating, the pellets were cured overnight in a ventilated oven at 40 C.

A plurality of pellets comprising 60 mg of duloxetine hydrochloride were subjected to dissolution testing. For the dissolution measurements, the following methods were used. USP 26, Physical Tests/{724} Drug Release, Delayed-release (enteric coated) articles-General drug release standard, Method B, pages 2160 and 2161. The dissolution test is based on USP [symbol] 711 [symbol] , “Apparatus 1”. Dissolution in pH 6.8 phosphate buffer USP, performed after 2 hrs dissolution in 0.1N HCl Method as described in text. Last 10 minutes final spin of 250 RPM basket speed. The results are shown in the FIGURE.

### EXAMPLE 2

### Composition

| | | Batch (%) |
|---|---|---|
| Core | Sugar spheres 600-710 microns | 45.9 |
| Drug layer | Duloxetine hydrochloride | 21.0 |
| | Methyl cellulose | 8.4 |
| Separating layer | HPMC Methocel E5 ™ | 2.2 |
| | Sucrose | 1.1 |
| | Talc | 1.1 |
| Enteric layer | Eudragit L100-55 | 9.6 |
| | Talc | 9.6 |
| | Dibutylsebacate | 1.1 |

| | |
|---|---|
| Content of the enteric layer: | 20.3% |

Process:

The pellet batch is prepared in a fluid-bed coating device (Aeromatic-Fielder MP-4/5) by bottom spray and with three Wurster columns installed.

Drug layer is applied onto inert sugar beads of a particle size 600-710 microns. The coating fluid is prepared by combining a dispersion of Duloxetine hydrochloride in water and a dispersion of methyl cellulose in water.

Separating layer is applied onto the so coated beads. The coating fluid is prepared by combining a dispersion of hypromellose in water with a dispersion of sucrose and talc in water (prepared by dissolution of sucrose in water followed by dispergating talc under mechanical stirring). The hypromellose is allowed to hydrate in water for at least one night.

Enteric coating layer is applied onto the beads with the above two coatings. The coating fluid is prepared by dissolving Eudragit in an isopropanol-water mixture (ratio 19:1), followed by addition of dibutylsebacate and dispergating of talc in the fluid.

After the coating, the pellets are cured overnight in a ventilated oven at 50° C.

Each of the patents, patent applications, and journal articles mentioned above are incorporated herein by reference. The invention having been described it will be obvious that the same may be varied in many ways and all such modifications are contemplated as being within the scope of the invention as defined by the following claims.

We claim:

1. A pharmaceutical dosage form comprising a plurality of pellets, wherein each pellet comprises:
i) a pellet core with a diameter of 600-1000 micrometer,
ii) a drug layer, comprising duloxetine or a pharmaceutical acceptable salt thereof and a binder,
iii) a separating layer, comprising a binder and a pore forming component, which constitutes 3-12 wt % of the total weight of the pellet composition, and
iv) an enteric coating layer, comprising a pharmaceutically acceptable acid resistant polymethacrylate polymer.

2. The pharmaceutical dosage form according to claim 1, wherein said drug layer is present in an amount between 24-32 wt % based on the total weight of the pellet composition.

3. The pharmaceutical dosage form according to claim 1, wherein said binder in said drug layer is methyl cellulose.

4. The pharmaceutical dosage form according to claim 1, wherein said enteric coating layer is present in an amount between 18-27 wt % based on the total weight of the pellet composition.

5. The pharmaceutical dosage form according to claim 1, wherein the polymethacrylate polymer is a methacrylic acid-ethyl acrylate co-polymer which constitutes 40-70 wt % of the enteric coating layer.

6. The pharmaceutical dosage form according to claim 5, wherein said enteric coating layer was applied with a non-aqueous liquid.

7. The pharmaceutical dosage form according to claim 6, wherein said enteric coating layer was applied as a solution in said non-aqueous liquid.

8. The pharmaceutical dosage form according to claim 1, wherein the pellet core is a sugar sphere.

9. The pharmaceutical dosage form according to claim 8, wherein said sugar sphere has a diameter within the range of 600-710 micrometers.

10. The pharmaceutical dosage form according to claim 1, wherein the duloxetine is duloxetine hydrochloride.

11. The pharmaceutical dosage form according to claim 1, wherein the binder in the separating layer is hydroxypropylmethylcellulose, polyvinylpyrrolidone, or a mixture of both, and wherein said binder constitutes 40-60 wt % of the total separating layer.

12. The pharmaceutical dosage form according to claim 11, wherein the pore forming component in the separating layer constitutes 10-40 wt % of the total separating layer.

13. The pharmaceutical dosage form according to claim 1, wherein the plurality of pellets exhibits a dissolution release profile of duloxetine of at least 75% release at 45 minutes in simulated intestinal fluid using USP [symbol] 711 [symbol] , Apparatus 1 method at 100 rpm after 2 hours of dissolution of the plurality of pellets in simulated gastric fluid using USP [symbol] 711 [symbol] , Apparatus 1 method at 100 rpm.

14. The pharmaceutical dosage form according to claim 1, wherein the plurality of pellets exhibits a dissolution release profile of duloxetine of at least 75% release at 45 minutes in simulated intestinal fluid using USP [symbol] 711 [symbol] , Apparatus 1 method at 100 rpm after 3 hours of dissolution of the plurality of pellets in simulated gastric fluid of pH 4.5 using USP [symbol] 711 [symbol] , Apparatus 1 method at 100 rpm.

15. The pharmaceutical dosage form according to claim 1, wherein the plurality of pellets exhibits a dissolution release profile of naphthol impurity of less the 1% after 2 hours in simulated gastric fluid using Ph. Eur. basket method at 100 rpm.

16. The pharmaceutical dosage form according to claim 1, wherein the plurality of pellets exhibits a dissolution release profile of naphthol impurity of less the 0.1% after 2 hours in simulated gastric fluid using USP [symbol] 711 [symbol] , Apparatus 1 method at 100 rpm.

17. The pharmaceutical dosage form according to claim 1, wherein the plurality of pellets exhibits a dissolution release profile of duloxetine of less the 5% after 3 hours in simulated gastric fluid of pH 4.5 using USP [symbol] 711 [symbol] , Apparatus 1 method at 100 rpm.

18. A method of treating which comprises administering the pharmaceutical dosage form according to claim 1, in an amount effective to treat stress urinary incontinence, major depressive disorder, generalized anxiety disorder, or neuropathic pain, to a patient in need thereof.

19. A process, which comprises:
coating a pellet core having a diameter of 600-1000 micrometer, with a drug layer, comprising duloxetine or a pharmaceutical acceptable salt thereof and a binder;
coating said drug layer with a separating layer, comprising a binder and a pore forming component, wherein said separating layer constitutes 3-12 wt % of the total weight of the pellet composition; and
coating said separating layer with an enteric coating layer, comprising a pharmaceutically acceptable acid resistant polymethacrylate polymer; wherein said enteric coating layer is coated as a solution in a non-aqueous solvent onto said separating layer.

20. The process according to claim 19, wherein the polymethacrylate polymer is a methacrylic acid-ethyl acrylate co-polymer which constitutes 40-70 wt % of the enteric coating layer; and wherein said non-aqueous solvent is an alcohol or an alcohol and water mixture.
